SEQUENCE LISTING (8 sequences)

SEQ ID NO: 1 - RNA, 5 nt, Artificial Sequence
  Synthetic Construct
auuua

SEQ ID NO: 2 - DNA, 13 bp, Artificial Sequence
  Synthetic Construct
tgyygatgyyyyy

SEQ ID NO: 3 - DNA, 6 bp, Artificial Sequence
  Synthetic Construct
aggagg

SEQ ID NO: 4 - DNA, 10 bp, Artificial Sequence
  Synthetic Construct
accaccatgg

SEQ ID NO: 5 - DNA, 10 bp, Artificial Sequence
  Synthetic Construct
gccaccatgg

SEQ ID NO: 6 - DNA, 29 bp, Artificial Sequence
  Synthetic Construct
tctctttacattttggtctctatactaca

SEQ ID NO: 7 - DNA, 738 bp, Artificial Sequence
  Synthetic Construct
atgtctaagggagaagagctgtttaccggcgtggtccctatcctggtggagctggacggcgatgtgaacggccagaaattcagcgtgtccggcgagggcgaaggcgacgccacctacggcaagctgacactgaagttcatctgcaccaccggcaagctgcctgtcccttggccaacactggtgaccaccttcagctacggagtgcaatgcttctccagataccctgaccacatgaagcagcacgatttctttaaatctgccatgcccgagggctacgtgcaggaacggaccatcttctacaaggacgacggaaattacaagaccagagccgaggtgaagttcgagggcgacaccctggtgaaccggatcgagctgaagggcatcgacttcaaagaggatggcaacatcctgggccacaagatggaatacaactacaactcccacaacgtgtacatcatggccgacaagcctaagaacggcatcaaggtgaacttcaagatcagacacaacatcaaggacggctctgtgcagctggccgaccactaccagcagaacacccccatcggcgacggccctgtgctgctgcctgataaccactatctgtctacacagtccgctctgtccaaagatcctaacgagaagcgggaccacatgatcctgctggagttcgtgaccgccgctggcatcacccatggcatggacgagctgtacaaggactacaaggacgatgacgacaag

SEQ ID NO: 8 - DNA, 1290 bp, Artificial Sequence
  Synthetic Construct
atgtccctgcactttctgtactactgctctgagcctaccctggacgtgaagatcgccttctgtcagggcttcgataagcaggtggacgtctcctatatcgctaagcactacaacatgagcaaatccaaggtggacaaccagttctactctgtcgaggtgggcgactctaccttcaccgtgctgaagagataccagaacctgaaacccatcggctccggcgctcagggcatcgtgtgcgccgcttacgacgccgtgctggatagaaacgtggccatcaagaagctgagccggcctttccagaaccagacacacgctaagcgggcctacagagagctggtcctgatgaagtgcgtgaaccacaagaacatcatctccctgctgaatgtgttcacccctcagaaaaccctggaagagttccaggatgtgtacctggtgatggaactgatggacgccaacctgtgccaggtgatccagatggaactggaccacgagcggatgtcctacctgctgtaccagatgctgtgtggcatcaagcacttgcatagcgctggcatcatccacagagatctgaaaccttctaacatcgtggtgaagtccgactgcaccctgaagatcctggacttcggcctggccagaaccgctggcacctctttcatgatgacaccctacgtggtgaccagatactaccgggcccctgaagtgatcctgggcatgggctacaaggagaacgtggacatctggtccgtgggatgcatcatgggcgagatggtcagacacaagatcctgttccccggaagagattacatcgaccagtggaacaaggtgatcgagcagctgggcaccccttgtcctgagttcatgaagaaactgcagcctaccgtgcggaactacgtggaaaaccggcctaagtacgccggcctgacctttccaaagctgttccctgactctctgttccccgctgacagcgagcacaacaagctgaaagcctctcaggccagagatctgctgtccaagatgctggtgatcgaccctgctaagagaatctccgtggacgatgccctgcagcacccctacatcaacgtgtggtacgaccctgctgaggtggaagcccctccacctcagatctacgacaagcagctggacgaaagagagcacaccatcgaggagtggaaggagctgatctataaagaagtgatgaactccgaggaaaagaccaagaacggcgtggtcaagggccagccttccccctctgctcaggtgcagcaagactacaaggacgatgatgacaag

(19) United States
(12) Patent Application Publication
(10) Pub. No.: US 2021/0366574 A1
(43) Pub. Date: Nov. 25, 2021

(54) CODON OPTIMIZATION

(71) Applicant: Nanjing Genscript Biotech Co., Ltd., Nanjing, Jiangsu (CN)

(72) Inventor: Long FAN, Nanjing (CN)

(21) Appl. No.: 17/257,208

(22) PCT Filed: Jul. 30, 2019
(86) PCT No.: PCT/CN2019/098258

§ 371 Date: Dec. 30, 2020

(30) Foreign Application Priority Data
Jul. 30, 2018 (CN) PCT/CN2018/097745

(51) Int. Cl. G16B 30/00; G16B 25/10; G16B 40/00; G16B 50/00

(52) U.S. Cl. CPC G16B 30/00; G16B 50/00; G16B 40/00; G16B 25/10

(57) ABSTRACT

An exemplary computer-implemented method for optimizing a nucleic acid sequence for expression of a protein in a host, comprises: a) receiving an initial population set, wherein the initial population set comprises a plurality of initial candidate nucleic acid sequences capable of expressing the protein (106); and b) performing, based on the initial population set, optimization of a harmony index, a codon context index, and an outlier index using a computer-assisted NS-GA-III algorithm or a variant thereof, thereby obtaining a plurality of optimized nucleic acid sequences capable of expressing the protein (108).

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 759892000440SEQLIST.TXT, date recorded: Jul. 25, 2018, size: 4 KB).

### FIELD OF INVENTION

The present disclosure relates generally to optimization techniques, and more specifically to systems and methods for optimizing a sequence (e.g., a nucleic acid sequence) for expression of a protein in a host.

### BACKGROUND

Codon degeneracy refers to the redundancy of the genetic code, which is exhibited as the phenomenon that an amino acid could be specified by different synonymous codons. Notably, it was discovered that these synonymous codons are used in unequal frequencies in most sequenced genomes. This phenomenon is termed codon-usage bias.

Since high-quality proteins with correct folding and modifications are required for biomedical and biotechnological research and industrial production, how to explore and summarize the potentially beneficial rules and patterns reflecting codon-usage bias of highly-expressed genes is essential for improving expression level of proteins. However, protein expression is a multi-step process involving regulation at the level of transcription, mRNA turnover, translation and post translational modifications enabling the formation of a stable product. Even a single synonymous codon substitution can increase the expression of a transgene by more than 1,000-fold. Thus, codon optimization is poised for the optimal expression of synthetic genes in the recombinant host.

### BRIEF SUMMARY

Provided herein are systems and methods for enhanced codon optimization that takes account of, as well as balances, a plurality of factors using a multi-objective optimization algorithm According to some embodiments, the codon optimization is based on, among other things, three objectives: (i) how to allocate the count of synonymous codons of certain amino acid at first, (ii) how to place a synonymous codon into its most suitable location, and (iii) how to reduce the adverse but accidentally generated subsequences and/or motifs. In some embodiments, these three objectives are quantified as the harmony index, the codon context index, and the outlier index. During optimization, the objectives are considered using a multi-objective algorithm such as the nondominated sorting genetic algorithm III (NSGA-III) or a variant thereof. Specifically, the objectives can be calculated, for a given candidate nucleic acid sequence, with reference to known characteristics of highly-expressed genes. In some embodiments, various known adverse motifs and/or features (e.g., as identified from literature) are removed from one or more optimized sequences before gene synthesis and protein expression.

Accordingly, the invention provides a systematic method whereby preferably all or most of the parameters and factors affecting protein expression including, but not limited to, codon harmony, codon usage (e.g., synonymous codon distribution), codon context index, cis-acting mRNA destabilizing motifs, RNase splicing sites, GC-content, ribosome binding site (RBS), mRNA secondary structure of the genes (e.g., mRNA free energy), and repetitive element are taken into consideration to improve and optimize the nucleic acid sequences to boost the protein expression of genes in expression systems, such as in expression host cells including both eukaryotic and prokaryotic cells such as mammalian, insect, yeast, bacterial, algal, and in cell-free expression system.

In some embodiments, there is provided a computer-implemented method for optimizing a nucleic acid sequence for expression of a protein in a host, comprising: a) receiving an initial population set, wherein the initial population set comprises a plurality of initial candidate nucleic acid sequences capable of expressing the protein; and b) performing, based on the initial population set, optimization of a harmony index, a codon context index, and an outlier index using a computer-assisted NSGA-III algorithm or a variant thereof, thereby obtaining a plurality of optimized nucleic acid sequences capable of expressing the protein, wherein the harmony index of a candidate nucleic acid sequence is indicative of consistency of usage frequency distribution of synonymous codons between a plurality of highly expressed genes and the candidate nucleic acid sequence, wherein the codon context index of the candidate nucleic acid sequence is a measure for placing a synonymous codon into a suitable location, and wherein the outlier index of the candidate nucleic acid sequence is a measure of negative effect of a plurality of predetermined sequence features on the candidate nucleic acid sequence.

In some embodiments, the method further comprises providing an output indicative of at least one optimized nucleic acid sequence of the plurality of optimized nucleic acid sequences.

In some embodiments, receiving an initial population set comprises: receiving a protein sequence; generating the initial population set based on the received protein sequence.

In some embodiments, receiving an initial population set comprises: receiving a nucleic acid sequence; translating the received nucleic acid sequence into a protein sequence; generating the initial population set based on the protein sequence.

In some embodiments, the initial population set is of a predetermined size.

In some embodiments, the initial population set includes binary representations of the plurality of initial candidate nucleic acid sequences.

In some embodiments, performing optimization of a harmony index, a codon context index, and an outlier index comprises: maximizing the harmony index; maximizing the codon context index; and minimizing the outlier index.

In some embodiments, performing optimization of a harmony index, a codon context index, and an outlier index comprises: calculating, for each initial candidate nucleic acid sequence of the initial population set, a respective harmony index value, a respective codon context index value, and a respective outlier index value for a respective initial candidate nucleic acid sequence; based on the calculating, assigning a plurality of fitness values corresponding to the plurality of initial candidate nucleic acid sequences; based on the plurality of fitness values, sorting the plurality of initial candidate nucleic acid sequences; and including a subset of the sorted plurality of initial candidate nucleic acid sequences in a subsequent population set. In some embodiments, the plurality of fitness values includes the harmony index, the codon context index, and the outlier index for the candidate nucleic acid sequence.

In some embodiments, the method further comprises generating an offspring population based on the initial population; and including the offspring population in the subsequent population set.

In some embodiments, the offspring population is generated via binary tournament selection, crossover/recombination, mutation, or any combination thereof.

In some embodiments, the initial population set and the subsequent population set are of the same size.

In some embodiments, performing optimization of a harmony index, a codon context index, and an outlier index comprises a plurality of iterations, wherein the i-th iteration of the plurality of iterations comprises: receiving a population set of nucleic acid sequences corresponding to the (i−1)th iteration; associating each nucleic acid sequence of the population set corresponding to the (i−1)th iteration with a non-domination level; sorting the nucleic acid sequences in the population set corresponding to the (i−1)th iteration based on the associated non-domination levels; generating a population set corresponding to the i-th iteration, wherein the population set corresponding to the i-th iteration includes a subset of the sorted nucleic acid sequences corresponding to the (i−1)th iteration and an offspring population generated based on the sorted nucleic acid sequences corresponding to the (i−1)th iteration; and determining, based on one or more terminating conditions, whether to proceed to a (i+1)th iteration using the population set corresponding to the i-th iteration.

In some embodiments, associating each nucleic acid sequence with a non-domination level comprises: calculating, for each nucleic acid sequence of the population set corresponding to the (i−1)th iteration, a respective harmony index value, a respective codon context index value, and a respective outlier index value.

In some embodiments, generating a population set corresponding to the i-th iteration comprises: associating at least one nucleic acid sequence of the sorted nucleic acid sequence corresponding to the (i−1)th iteration with one of a plurality of predetermined reference points.

In some embodiments, the one or more terminating conditions includes: a fixed number of iterations reached, best fitness reached a plateau and no better results produced, a minimum criteria of near-optimal solution satisfied by some solutions, or any combination thereof.

In some embodiments, the harmony index of a candidate nucleic acid sequence is calculated based on a formula: H=1−D(Fₕₛ, Fₜₛ), wherein D( ) indicates a distance function; wherein Fₕₛ includes a vector comprising frequencies of synonymous codons of a plurality of amino acids within a plurality of highly expressed genes; and wherein Fₜₛ includes a vector comprising of frequencies of synonymous codons of the plurality of amino acids within a coding gene of the candidate nucleic acid sequence.

In some embodiments, D( ) indicates a function measuring a distance between two vectors. In some embodiments, D( ) is a distance function that includes, but is not limited to: Euclidean distance, a Cosine distance, a Manhattan distance, or a Minkowski distance of two vectors.

In some embodiments, a frequency of a synonymous codon of the plurality of highly expressed genes or a candidate nucleic acid sequence is defined as:

$${F}_{{s}_{ij}} = \frac{total occurancy of synonymous codon j}{total occurancy of amino acid i} , ∀ i ∈ \{ A , C , D , E , F , G , H , I , K , L , N , P , Q , R , S , T , V , Y \} and ∃ j ∈ 59 synonymous codons .$$

In some embodiments, the codon context index of a candidate nucleic acid sequence is calculated based on a formula: CC=1−D(F_{hcc}, F_{tcc}), wherein D( ) indicates a distance function; wherein F_{hcc} comprises a vector comprising frequencies of synonymous codon pairs of two continual amino acids within a plurality of highly expressed genes; and wherein F_{tcc} comprises a vector comprising frequencies of synonymous codon pairs of two continual amino acids within a coding gene of the candidate nucleic acid sequence.

In some embodiments, D( ) indicates a function measuring a distance between two vectors. In some embodiments, D( ) is a distance function that includes, but is not limited to: Euclidean distance, a Cosine distance, a Manhattan distance, or a Minkowski distance of two vectors.

In some embodiments, a frequency of a synonymous codon pair of the plurality of highly expressed genes or a candidate nucleic acid sequence is defined as:

$${F}_{{cc}_{ij}} = \frac{total occurancy of synonymous codon pair j}{total occurancy of amino acid pair i} , ∀ i ∈ the permutation of two amino acids besides MM , MW , WW and WM \text{;} ∃ j ∈ 3717 codon pairs .$$

In some embodiments, the outlier index is calculated based on a formula: O=Σᵢ₌₁^{N} wᵢ×fᵢ(x), wherein N is the number of the plurality of predetermined sequence features; wherein fi(x) denotes a penalty scoring function of the ith sequence feature of the plurality of predetermined sequence features; and wherein wi denotes a relative weight associated with fi(x).

In some embodiments, the plurality of predetermined features includes: GC-content value, CIS elements, repetitive elements, RNA splicing sites, ribosome binding sequences, minimal free energy of mRNA, or any combination thereof.

In some embodiments, the plurality of predetermined features is identified based on a selected expression system.

In some embodiments, a variant of the NSGA-III algorithm includes the EliteNSGA-III algorithm or a NSGA-II based immune algorithm.

In some embodiments, performing optimization of a harmony index, a codon context index, and an outlier index comprises: ranking the plurality of optimized nucleic acid sequences by descending order of harmony index, then by descending order of codon context index, and then by ascending order of outlier index; selecting one or more top-ranked optimized nucleic acid sequences for synthesis.

In some embodiments, the method further comprises: c) removing a predetermined adverse subsequence or motif from an optimized nucleic acid sequence of the plurality of optimized nucleic acid sequences.

In some embodiments, the predetermined adverse subsequence or motif is identified based on analysis of a plurality of text portions.

In some embodiments, removing the predetermined adverse subsequence or motif comprises: identifying the predetermined adverse subsequence or motif in the optimized nucleic acid sequence; identifying a plurality of synonymous codons based on identified predetermined adverse subsequence or motif; selecting a synonymous codon from the plurality of synonymous codons for substitution with the identified predetermined adverse subsequence in the optimized nucleic acid sequence.

In some embodiments, at least one of the harmony index, the codon context index, and the outlier index is calculated based on one or more characteristics of a plurality of highly-expressed genes from one or more databases.

In some embodiments, the one or more characteristics include codon frequency, synonymous codon frequency, codon pair frequency, or a combination thereof.

In some embodiments, the method further comprises setting one or more parameters, wherein the one or more parameters include a size of a population set, a number of divisions, a distribution index for simulated binary crossover, a crossover rate for simulated binary crossover, a mutation rate for bit flip mutation, a distribution index for bit flip mutation, or any combination thereof.

In some embodiments, there is provided a non-transitory computer-readable storage medium storing one or more programs, the one or more programs comprising instructions, which when executed by one or more processors of an electronic device, cause the electronic device to carry out any of the methods described herein.

In some embodiments, there is provided a system for optimizing a nucleic acid sequence for expression of a protein in a host, the system comprising: one or more processors; a memory; and one or more programs, wherein the one or more programs are stored in the memory and configured to be executed by the one or more processors, the one or more programs including instructions for carrying out any of the methods described herein.

In some embodiments, there is provided an electronic device for optimizing a nucleic acid sequence for expression of a protein in a host, the device comprising means for carrying out any of the methods described herein.

In some embodiments, there is provided a program product stored on a recordable medium for optimizing a nucleic acid sequence for expression of a protein in a host, the program product comprising a computer software for carrying out any of the methods described herein.

In some embodiments, there is provided an isolated nucleic acid molecule comprising the optimized nucleic acid sequence obtained from any of the methods described herein.

In some embodiments, there is provided a vector comprising the above-mentioned isolated nucleic acid molecule.

In some embodiments, there is provided a recombinant host cell comprising the above-mentioned isolated nucleic acid molecule or the above-mentioned vector.

In some embodiments, there is provided a method for expressing a protein in a host cell, the method comprising: (a) obtaining an optimized nucleic acid sequence for expression of the protein in the host cell using any of the methods described herein, (b) synthesizing a nucleic acid molecule comprising the optimized nucleic acid sequence; (c) introducing the nucleic acid molecule into the host cell to obtain a recombinant host cell; and (d) cultivating the recombinant host cell under conditions to allow expression of the protein from the optimized nucleic acid sequence.

### DESCRIPTION OF THE FIGURES

FIG. 1 depicts a block diagram of an exemplary process for codon optimization, in accordance with some embodiments.

FIG. 2A depicts an exemplary pipeline for constructing and executing an algorithm for optimizing a sequence (e.g., a nucleic acid sequence) for expression of a protein in a host, in accordance with some embodiments.

FIG. 2B depicts an exemplary general workflow of genetic algorithm, in accordance with some embodiments.

FIG. 3 depicts Western blot result of optimized GFP and JNK3A1 relative to their wild type, in accordance with some embodiments.

FIG. 4 depicts an exemplary electronic device, in accordance with some embodiments.

### DETAILED DESCRIPTION

The present invention provides enhanced codon optimization for improving the recombinant expression of genes in various host, including but not limited to E. coli, CHO, HEK293, yeast, insect, cell-free expression system, etc. An exemplary system according to the present invention collects highly-expressed genes for an expression system, extracts basic sequence features, duplicates the beneficial comprehensive patterns in the sequence of interest (e.g., a nucleic acid sequence), and remove adverse features so as to improve the expression of target genes at the expression system.

Currently, a number of tools of codon optimization have been developed and are summarized below in Table 1. Multiple, preferably most or all, of the parameters and factors including codon usage (e.g., Codon Adaptation Index [CAI], Effective Number of codons [ENc], Relative Synonymous Codon Usage [RSCU] and Synonymous Codon Usage Order [SCUO]), codon pair, tRNA usage (e.g., tRNA adaptation index [tAI]), GC-content, ribosome binding site (RBS), hidden stop codons, motif avoidance, restriction site removal, mRNA secondary structure of the genes (e.g., mRNA free energy) and hydropathy index optimization, have been taken into consideration by these tools so as to boost the expression during codon optimization of bacteria, yeast, insect and mammalian cells.

TABLE 1

| Gene design tool | Web URL |
|---|---|
| DNAWorks | http://helixweb.nih.gov/dnaworks/ |
| Jcat | http://www.jcat.de/ |
| Syntheticgenedesigner | http://userpages.umbc.edu/~wug1/codon/sgd/ |
| GeneDesign | http://genedesign.org/ |
| Gene Designer2.0 | http://www.dna20.com/resources/genedesigner |
| OPTIMIZER | http://genomes.urv.es/OPTIMIZER |
| Visualgenedeveloper | http://www.visualgenedeveloper.net/ |
| Eugene | http://bioinformatics.ua.pt/eugene |
| mRNA Optimizer | http://bioinformatics.ua.pt/software/mRNA- |
| | optimiser |
| COOL | http://bioinfo.bti.a-star.edu.sg/COOL/ |
| D-Tailor | http://sourceforge.net/projects/dtailor/ |

However, because so many factors could be considered to the key points, how to balance them remains a challenge since this is a multiple objective optimization problem but the objectives may be conflicting with each other. On the other hand, omitting one or more factors or parameters from the consideration may result in low or no expression of the target genes in expression systems.

Provided herein are systems and methods for enhanced codon optimization that takes account of, as well as balances, a plurality of factors using a multi-objective optimization algorithm According to some embodiments, the codon optimization is based on, among other things, three objectives: (i) how to allocate the count of synonymous codons of certain amino acid at first, (ii) how to place a synonymous codon into its most suitable location, and (iii) how to reduce the adverse but accidentally generated subsequences and/or motifs. In some embodiments, these three objectives are quantified as the harmony index, the codon context index, and the outlier index. During optimization, the objectives are considered using a multi-objective algorithm such as the nondominated sorting genetic algorithm III (NSGA-III) or a variant thereof. Specifically, the objectives can be calculated, for a given candidate nucleic acid sequence, with reference to known characteristics of highly-expressed genes. In some embodiments, various known adverse motifs and/or features (e.g., as identified from literature) are removed from one or more optimized sequence before gene synthesis and protein expression.

Accordingly, the invention provides a systematic method whereby preferably all or most of the parameters and factors affecting protein expression including, but not limited to, codon harmony, codon usage (e.g., synonymous codon distribution), codon context index, cis-acting mRNA destabilizing motifs, RNase splicing sites, GC-content, ribosome binding site (RBS), mRNA secondary structure of the genes (e.g., mRNA free energy), and repetitive element are taken into consideration to improve and optimize the nucleic acids to boost the protein expression of genes in expression systems, such as in expression host cells including both eukaryotic and prokaryotic cells such as mammalian, insect, yeast, bacterial, algal, and in cell-free expression system.

Thus, the present invention in one aspect provides for methods for sequence optimization for improved recombinant protein expression using a NSGA-III algorithm or its variants to optimize multiple (e.g., more than 2) objectives. In another aspect, there are provided methods for removing adverse motifs and features from the nucleic acid sequence (e.g., after the iterations of the NSGA-III algorithms are completed) before gene synthesis and protein expression. Also provided are methods for quantifying and calculating the multiple objectives in the optimization algorithms, as well as methods for identifying adverse motifs and features to reduce or remove.

Also provided are systems, non-transitory computer-readable storage medium, electronic devices, and program products for storing one or more programs for carrying out any one or more steps of the methods described herein. Also provided are isolated nucleic acid molecules comprising the optimized nucleic acid sequences obtained from the methods described herein; vectors comprising said isolated nucleic acid molecules; recombinant host cells comprising said isolated nucleic acid molecule or said vector. Also provided are methods for expressing a protein in a host cell involving any of the methods described herein.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “or,” and “the” include plural referents unless the context clearly dictates otherwise.

As used herein and in the appended claims, “set” refers to one or a plurality of referents unless the context clearly dictates otherwise.

Methods of Codon Optimization

The present invention in one aspect provides for methods (e.g., computer-implemented or computer-assisted methods) for optimizing a nucleic acid sequence for expression of a protein in a host. Related for these methods are methods for removing adverse motifs and features from the nucleic acid sequence (e.g., after the iterations of the NSGA-III algorithms are completed) before gene synthesis and protein expression. Also related to these methods are methods for quantifying and calculating the multiple objectives in the optimization algorithms, as well as methods for identifying adverse motifs and features to reduce or remove.

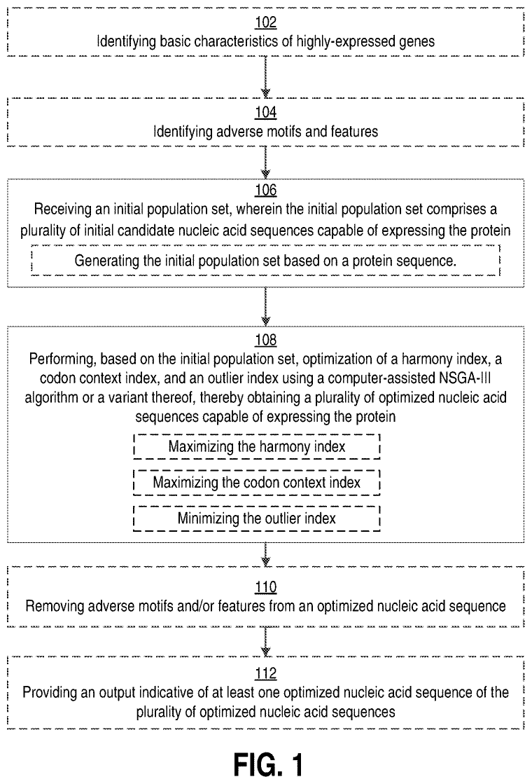

FIG. 1 illustrates an exemplary process 100 for codon optimization, with dash blocks denoting optional steps. While portions of process 100 are described herein as being performed by particular devices, it will be appreciated that process 100 is not so limited. In other examples, process 100 is performed using only a single electronic device (e.g., electronic device 400) or multiple electronic devices. In process 100, some blocks are, optionally, combined, the order of some blocks is, optionally, changed, and some blocks are, optionally, omitted. In some examples, additional steps may be performed in combination with the process 100.

At block 106, an electronic device receives an initial population set, wherein the initial population set comprises a plurality of initial candidate nucleic acid sequences capable of expressing the protein. In some embodiments, the initial population set is randomly generated. In some embodiments, the initial population set is of a predetermined size (e.g., determined by a user).

In some embodiments, as shown in block 106, receiving an initial population set includes generating the initial population set based on a protein sequence. For example, receiving an initial population set can include: receiving a protein sequence (e.g., as an input from a user); and generating the initial population set based on the received protein sequence. As another example, receiving an initial population set can include: receiving a nucleic acid sequence (e.g., as an input from the user); translating the received nucleic acid sequence into a protein sequence; generating the initial population set based on the protein sequence.

In some embodiments, the initial population set includes binary representations (e.g., binary strings) of the plurality of initial candidate nucleic acid sequences. Generally, binary string, but not codon list/array/vector, is selected as data structure to denote coding gene, and all operation objects of the genetic algorithm including population initialization, crossover/recombination, mutation, selection are binary strings except the fitness evaluation of genes before selection. As described further below, in some embodiments, when fitness functions (i.e., three index functions) need to be evaluated for each individual of the whole population before selection, the binary representations should be transformed back into codon strings temporally.

At block 108, the electronic device performs, based on the initial population set, optimization of a harmony index, a codon context index, and an outlier index using a computer-assisted NSGA-III algorithm or a variant thereof, thereby obtaining a plurality of optimized nucleic acid sequences capable of expressing the protein.

Always, or in some embodiments, the harmony index of a candidate nucleic acid sequence is indicative of consistency of usage frequency distribution of synonymous codons between a plurality of highly expressed genes and the candidate nucleic acid sequence (i.e., gene encoding candidate protein during optimization), which helps to solve how to allocate the count of synonymous codons of certain amino acid. The codon context index of the candidate nucleic acid sequence is a measure for placing a synonymous codon into a suitable location. The outlier index of the candidate nucleic acid sequence is a measure of negative effect of a plurality of predetermined sequence features on the candidate nucleic acid sequence.

In some embodiments, as shown in block 106, performing optimization of a harmony index, a codon context index, and an outlier index comprises: maximizing the harmony index; maximizing the codon context index; and minimizing the outlier index.

The optimization can be performed by using a multi-objective genetic algorithm, the three objectives being maximizing the harmony index; maximizing the codon context index; and minimizing the outlier index. In some embodiments, the NSGA-III algorithm or a variant is used. Unlike traditional genetic algorithm, the maintenance of diversity among population members in NSGA-III is aided by supplying and adaptively updating a number of well-spread predefined reference points, thus NSGA-III have significant changes in its selection operator. Further, NSGA-III demonstrates its efficacy in solving three-objective to 15-objective optimization problems relative to other genetic algorithms, like NSGA-II. A variant of the NSGA-III algorithm includes the EliteNSGA-III algorithm, a NSGA-II based immune algorithm, MAM-MOIA or MOLA. The EliteNSGA-III algorithm is described in a publication titled “ELITENSGA-III: AN IMPROVED EVOLUTIONARY MANY-OBJECTIVE OPTIMIZATION ALGORITHM” by Amin Ibrahim et al., published in 2016, which is incorporated herein by reference in its entirety. Various immune algorithms are described in, for example, a publication titled “MOIA: MULTI-OBJECTIVE IMMUNE ALGORITHM” by Guan-Chun Luh et al., published in September 2010, a publication titled “OVERVIEW OF ARTIFICIAL IMMUNE SYSTEMS FOR MULTI-OBJECTIVE OPTIMIZATION” by Felipe Campelo et al., published in 2007, a publication titled “A MULTIOBJECTIVE IMMUNE ALGORITHM BASED ON A MULTIPLE-AFFINITY MODEL” by Zhi-Hua Hu, published in April 2010, and Chinese Patent Application No. 201710611752.5, filed on Jul. 25, 2017, which are incorporated herein by reference in their entireties.

In accordance with the operation of the NSGA-III algorithm (or similar genetic algorithms), performing optimization of a harmony index, a codon context index, and an outlier index comprises: calculating, for each initial candidate nucleic acid sequence of the initial population set, a respective harmony index value, a respective codon context index value, and a respective outlier index value for a respective initial candidate nucleic acid sequence; based on the calculating, assigning a plurality of fitness values corresponding to the plurality of initial candidate nucleic acid sequences; based on the plurality of fitness values, sorting the plurality of initial candidate nucleic acid sequences; and including a subset of the sorted plurality of initial candidate nucleic acid sequences in a subsequent population set (i.e., to be used in the 2^{nd} iteration).

In accordance with the operation of the NSGA-III algorithm (or similar genetic algorithms), the method further comprises generating an offspring population based on the initial population; and including the offspring population in the subsequent population set (i.e., to be used in the 2^{nd} iteration). In some embodiments, the offspring population is generated via binary tournament selection, crossover/recombination, mutation, or any combination thereof.

In some embodiments, the initial population set and the subsequent population set (i.e., to be used in the 2^{nd} iteration) are of the same size.

In accordance with the operation of the NSGA-III algorithm (or similar genetic algorithms), performing optimization of a harmony index, a codon context index, and an outlier index comprises a plurality of iterations. The i-th iteration of the plurality of iterations (wherein i can be 2, 3, 4, 5, 6 . . . n) comprises: receiving a population set of nucleic acid sequences corresponding to the (i−1)th iteration; associating each nucleic acid sequence of the population set corresponding to the (i−1)th iteration with a non-domination level; sorting the nucleic acid sequences in the population set corresponding to the (i−1)th iteration based on the associated non-domination levels; generating a population set corresponding to the i-th iteration, wherein the population set corresponding to the i-th iteration includes a subset of the sorted nucleic acid sequences corresponding to the (i−1)th iteration and an offspring population generated based on the sorted nucleic acid sequences corresponding to the (i−1)th iteration; and determining, based on one or more terminating conditions, whether to proceed to a (i+1)th iteration using the population set corresponding to the i-th iteration.

In some embodiments, associating each nucleic acid sequence with a non-domination level comprises: calculating, for each nucleic acid sequence of the population set corresponding to the (i−1)th iteration, a respective harmony index value, a respective codon context index value, and a respective outlier index value.

In accordance with the operation of the NSGA-III algorithm, in some embodiments, generating a population set corresponding to the i-th iteration comprises: associating at least one nucleic acid sequence of the sorted nucleic acid sequence corresponding to the (i−1)th iteration with one of a plurality of predetermined reference points.

In some embodiments, the one or more terminating conditions includes: a fixed number of iterations reached, best fitness reached a plateau and no better results produced, a minimum criteria of near-optimal solution satisfied by some solutions, or any combination thereof.

In some embodiments, the method further comprises setting one or more parameters for the optimization algorithm, wherein the one or more parameters include a size of a population set, a number of divisions, a distribution index for simulated binary crossover, a crossover rate for simulated binary crossover, a mutation rate for bit flip mutation, a distribution index for bit flip mutation, or any combination thereof.

In some embodiments, during optimization, at least one of the harmony index, the codon context index, and the outlier index is calculated based on one or more characteristics of a plurality of highly-expressed genes from one or more databases. In some embodiments, the one or more characteristics include codon frequency, synonymous codon frequency, codon pair frequency, or a combination thereof. These characteristics of highly-expressed genes can be used to calculate the harmony index, the codon context index, and the outlier index, for a given candidate nucleic acid sequence as shown by the formulas below.

In some embodiments, as indicated in block 102, these characteristics of highly-expressed genes are identified based on private or public databases. For example, the database(s) can be a proprietary database comprising previously successfully optimized orders collected from the order system of a company. As another example, the data can be obtained by way of data mining of RNA-seq data under various culture conditions, which may be public information. Data processing is performed with the aim to get the basic information of highly-expressed genes including codon frequency, synonymous codon frequency and codon pair frequency.

In some embodiments, the harmony index of a candidate nucleic acid sequence is calculated based on a formula: H=1−D (Fₕₛ, Fₜₛ), wherein D( ) indicates a distance function; wherein Fₕₛ includes a vector comprising frequencies of synonymous codons of a plurality of amino acids within a plurality of highly expressed genes; and wherein Fₜₛ includes a vector comprising of frequencies of synonymous codons of the plurality of amino acids within a coding gene of the candidate nucleic acid sequence.

In some embodiments, D( ) indicates a function measuring a distance between two vectors. In some embodiments, D( ) is a distance function that includes, but is not limited to: Euclidean distance, a Cosine distance, a Manhattan distance, or a Minkowski distance of two vectors.

In some embodiments, a frequency of a synonymous codon of the plurality of highly expressed genes or a candidate nucleic acid sequence is defined as:

$${F}_{{s}_{ij}} = \frac{total occurancy of synonymous codon j}{total occurancy of amino acid i} , ∀ i ∈ \{ A , C , D , E , F , G , H , I , K , L , N , P , Q , R , S , T , V , Y \} and ∃ j ∈ 59 synonymous codons .$$

In some embodiments, the codon context index of a candidate nucleic acid sequence is calculated based on a formula: CC=1−D(F_{hcc}, F_{tcc}), wherein D( ) indicates a distance function; wherein F_{hcc} comprises a vector comprising frequencies of synonymous codon pairs of two continual amino acids within a plurality of highly expressed genes; and wherein F_{tcc} comprises a vector comprising frequencies of synonymous codon pairs of two continual amino acids within a coding gene of the candidate nucleic acid sequence.

In some embodiments, D( ) indicates a function measuring a distance between two vectors. In some embodiments, D( ) is a distance function that includes, but is not limited to: Euclidean distance, a Cosine distance, a Manhattan distance, or a Minkowski distance of two vectors.

In some embodiments, a frequency of a synonymous codon pair of the plurality of highly expressed genes or a candidate nucleic acid sequence is defined as:

$${F}_{{cc}_{ij}} = \frac{total occurancy of synonymous codon pair j}{total occurancy of amino acid pair i} , ∀ i ∈ the permutation of two amino acids besides MM , MW , WW and WM \text{;} ∃ j ∈ 3717 codon pairs .$$

In some embodiments, the outlier index is calculated based on a formula: O=Σᵢ₌₁^{N} wᵢ×fᵢ(x), wherein N is the number of the plurality of predetermined sequence features; wherein fi(x) denotes a penalty scoring function of the ith sequence feature of the plurality of predetermined sequence features; and wherein wi denotes a relative weight associated with fi(x).

In some embodiments, the plurality of predetermined features includes: GC-content value, CIS elements, repetitive elements, RNA splicing sites, ribosome binding sequences, minimal free energy of mRNA, or any combination thereof.

In some embodiments, the plurality of predetermined features is identified based on a selected expression system. For various expression systems, the catalogues of adverse factors may change, of which the impacts or weights are also unequal.

In some embodiments, performing optimization of a harmony index, a codon context index, and an outlier index comprises: ranking the plurality of optimized nucleic acid sequences by descending order of harmony index, then by descending order of codon context index, and then by ascending order of outlier index; selecting one or more top-ranked optimized nucleic acid sequences for synthesis.

At block 110, the method optionally further comprises: c) removing a predetermined adverse subsequence or motif from an optimized nucleic acid sequence of the plurality of optimized nucleic acid sequences. In some embodiments, removing the predetermined adverse subsequence or motif comprises: identifying the predetermined adverse subsequence or motif in the optimized nucleic acid sequence; identifying a plurality of synonymous codons based on identified predetermined adverse subsequence or motif; selecting a synonymous codon from the plurality of synonymous codons for substitution with the identified predetermined adverse subsequence in the optimized nucleic acid sequence.

In some embodiments, the predetermined adverse subsequence or motif is identified based on analysis of a plurality of text portions (e.g., automatic text mining or manual checking of literature), as indicated in block 104.

In some embodiments, the method further comprises providing an output indicative of at least one optimized nucleic acid sequence of the plurality of optimized nucleic acid sequences.

In some embodiments, there is provided a non-transitory computer-readable storage medium storing one or more programs, the one or more programs comprising instructions, which when executed by one or more processors of an electronic device, cause the electronic device to carry out any of the methods described herein.

In some embodiments, there is provided a system for optimizing a nucleic acid sequence for expression of a protein in a host, the system comprising: one or more processors; a memory; and one or more programs, wherein the one or more programs are stored in the memory and configured to be executed by the one or more processors, the one or more programs including instructions for carrying out any of the methods described herein.

In some embodiments, there is provided an electronic device for optimizing a nucleic acid sequence for expression of a protein in a host, the device comprising means for carrying out any of the methods described herein.

In some embodiments, there is provided a program product stored on a recordable medium for optimizing a nucleic acid sequence for expression of a protein in a host, the program product comprising a computer software for carrying out any of the methods described herein.

In some embodiments, there is provided an isolated nucleic acid molecule comprising the optimized nucleic acid sequence obtained from any of the methods described herein.

In some embodiments, there is provided a vector comprising the above-mentioned isolated nucleic acid molecule.

In some embodiments, there is provided a recombinant host cell comprising the above-mentioned isolated nucleic acid molecule or the above-mentioned vector.

In some embodiments, there is provided a method for expressing a protein in a host cell, the method comprising: (a) obtaining an optimized nucleic acid sequence for expression of the protein in the host cell using any of the methods described herein, (b) synthesizing a nucleic acid molecule comprising the optimized nucleic acid sequence; (c) introducing the nucleic acid molecule into the host cell to obtain a recombinant host cell; and (d) cultivating the recombinant host cell under conditions to allow expression of the protein from the optimized nucleic acid sequence.

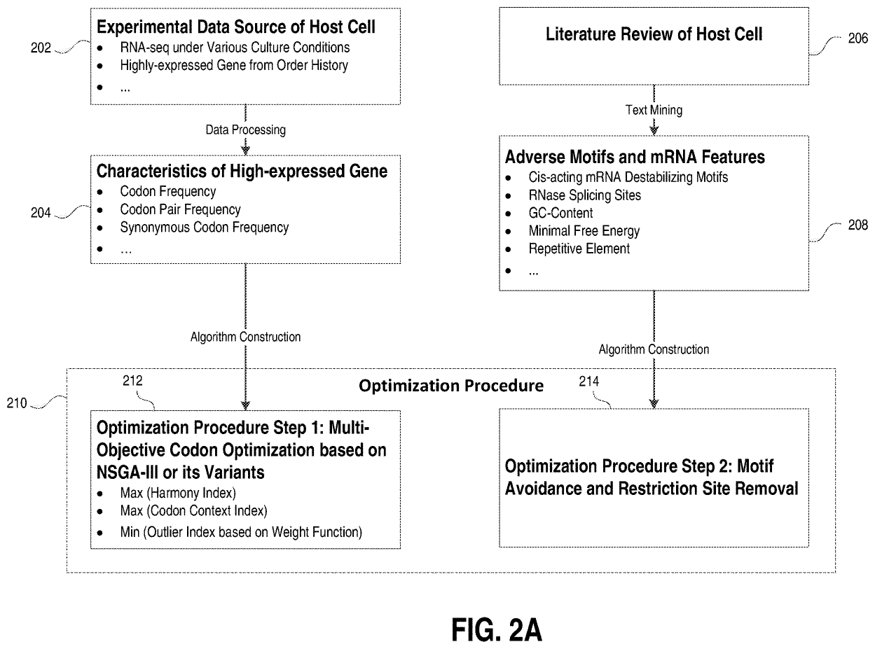

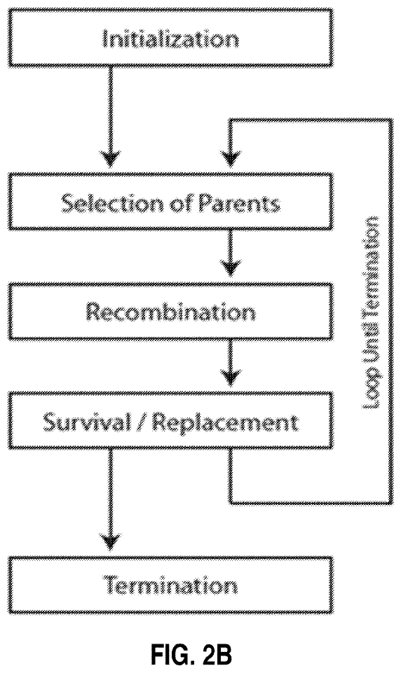

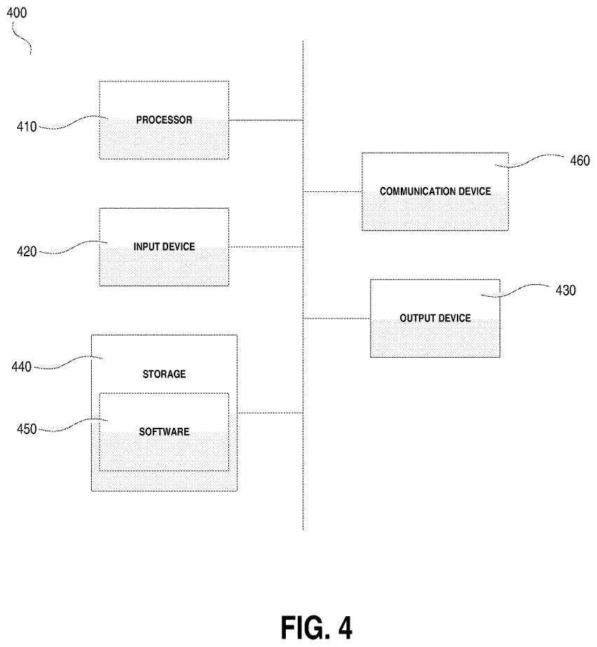

FIG. 2A illustrates an exemplary pipeline 200 for constructing and executing an algorithm for optimizing a sequence (e.g., a nucleic acid sequence) for expression of a protein in a host, according to some embodiments of the invention. Process 200 is performed, for example, using one or more electronic devices illustrated in FIG. 4. In some examples, process 200 is performed using a client-server system, and the blocks of process 200 are divided up in any manner between the server and a client device. In other examples, the blocks of process 200 are divided up between the server and/or multiple client devices. Thus, while portions of process 200 are described herein as being performed by particular devices, it will be appreciated that process 200 is not so limited. In other examples, process 200 is performed using only a single electronic device (e.g., electronic device 400) or multiple electronic devices. In process 200, some blocks are, optionally, combined, the order of some blocks is, optionally, changed, and some blocks are, optionally, omitted. In some examples, additional steps may be performed in combination with the process 200.

Data Collection and Literature Review

With reference to FIG. 2A, at block 202, a plurality of highly-expressed genes can be identified from one or more databases. The databases can be public or private. For example, the database(s) can be a proprietary database comprising previously successfully optimized orders collected from the order system of a company. As another example, the data can be obtained by way of data mining of RNA-seq data under various culture conditions, which may be public information.

At block 204, basic characteristics of the highly-expressed genes are identified. In an exemplary implement, mRNA-seq experiments and data analysis are performed following Illumina's recommended mRNA-Seq workflow for standard samples. During the course, TruSeq Stranded mRNA Library Prep Kit can be used for library preparation, and PE300 of NextSeq can be utilized for sequencing. Subsequently, data processing through TopHat, Cufflinks and home-made scripts can be applied with the aim to get the basic information of highly-expressed genes including codon frequency, synonymous codon frequency and codon pair frequency.

At blocks 206 and 208, the exemplary system can also identify any reported and validated adverse features to avoid in order to maintain the established advantages. To discover negative factors that may result in reduction of protein expression, the system can conduct literature review. For example, by way of automatic text mining and/or manual checking, the reported expression-related adverse motifs and mRNA features can be identified for various hosts.

Key Factors/Fitness Functions for the Optimization Algorithm

The expression of coding gene has multiple steps, which depends on the level of transcription, mRNA turnover, translation (including initiation, promoter escaping, elongation and termination) and post translational modifications. Nevertheless, codon optimization can be simplified as a combinational problem and grouped into three intuitive manipulations: (i) how to allocate the count of synonymous codons of certain amino acid at first, (ii) how to place a synonymous codon into its most suitable location, and (iii) how to reduce the adverse but accidentally generated subsequences and/or motifs.

In accordance with some embodiments of the invention, provided below are three key factors that match the three above-mentioned manipulations respectively and are highly correlative with protein expression: the harmony index, the codon context index, and the outlier index. As discussed below, these three indices are calculated based on the above-mentioned foundational data collected from various data sources.

With reference to FIG. 2A, at block 210, an optimization procedure comprising two steps 212 and 214 are carried out. At step 1 shown in block 212, the system performs multi-objective codon optimization based on the NSGA-III algorithm or its variants, which involves maximizing the harmony index, maximizing the codon context index, and minimizing the outlier index.

1. Harmony Index

Harmony index represents the consistency of usage frequency distribution of synonymous codons between highly expressed genes and a candidate nucleic acid sequence. The candidate nucleic acid sequence refers to a gene encoding candidate protein evaluated in at least one iteration of an optimization algorithm, which is described in detail under heading “Multi-Objective Optimization Algorithm”. In some embodiments, harmony index is defined as:

H=1−D(Fₕₛ,Fₜₛ)

In the formula above, H is harmony index, and D( ) is a distance function between two vectors which can be but is not limited to: Euclidean distance, Cosine distance, Manhattan distance, or Minkowski distance. Fₕₛ is a vector comprising of frequencies of synonymous codons of 18 amino acids (except Met/M and Trp/W) within highly expressed genes, and has 59 elements due to the removal of three stop codons (i.e., TAA, TAG and TGA), the codon of amino acid Met/M (i.e., ATG), and the codon of amino acid Trp/W (i.e., TGG) from 64 codons. Fₜₛ is a vector comprising frequencies of synonymous codons of 18 amino acids within the coding gene of candidate protein waiting for codon optimization (i.e., the candidate nucleic acid sequence).

Relative to the codon adaptation index (CAI), harmony index concentrates on the distribution (i.e., usage balancing/load balancing) of synonymous codons but does not always aim to maximum CAI through selecting uniquely Top 1 synonymous codon that occurs most frequently.

In some embodiments, frequency of certain synonymous codon of highly expressed genes or candidate nucleic acid sequence used during the calculation of harmony index is defined as:

$${F}_{{s}_{ij}} = \frac{total occurancy of synonymous codon j}{total occurancy of amino acid i} , ∀ i ∈ \{ A , C , D , E , F , G , H , I , K , L , N , P , Q , R , S , T , V , Y \} and ∃ j ∈ 59 synonymous codons .$$

Although harmony index takes the codon usage into consideration, it only cares about the frequency distribution of synonymous codons, while their allocation at different loci of one of 18 amino acids is still a problem (i.e., ordering setting of synonymous codons of the same amino acid). Thus, codon context index described below is required for solving this bottleneck through synonymous codon pairing to choose the approximately optimal ranking for the synonymous codon.

2. Codon Context Index

The codon context index of the candidate nucleic acid sequence is a measure for placing a synonymous codon into a suitable location. In some embodiments, the codon context index is defined as:

CC=1−D(F_{hcc},F_{tcc}).

In the formula above, CC stands for codon context index, and D( ) is a distance function between two vectors which can be but is not limited to: Euclidean distance, Cosine distance, Manhattan distance, or Minkowski distance. F_{hcc} is a vector comprising of frequencies of synonymous codon pairs of all kinds of two continual amino acids within highly expressed genes. For instance, amino acid Phe/F has two synonymous codons, i.e., TTT and TTC; and amino acid Lys/K has AAA and AAG as codons as well; their synonymous codon pairs should be 2 by 2 combinations including TTTAAA, TTTAAG, TTCAAA and TTCAAG. Since no synonymous codon pair exists for permutation of two amino acids methionine/M and tryptophan/W (i.e., MM, MW, WW and WM), the length of CC is 61 by 61 minus 4 and finally equals to 3717. F_{tcc} is a vector comprising of frequencies of synonymous codon pairs of all kinds of two continual amino acids within the coding gene of candidate protein (i.e., the candidate nucleic acid sequence), of which the length is 3717 as well.

Frequency of certain synonymous codon pair of highly expressed genes or candidate nucleic acid sequence used during the calculation of codon context index is defined as:

$${F}_{{cc}_{ij}} = \frac{total occurancy of synonymous codon pair j}{total occurancy of amino acid pair i} , ∀ i ∈$$

the permutation of two amino acids besides MM, MW, WW and WM; ∃j∈3717 codon pairs.

3. Outlier Index

Outlier index is a measure calculated by a weighted function to evaluate the negative effects of the identified plurality of sequence features on protein expression. In some embodiments, the outlier index is defined as:

$$0 = {\sum }_{i = 1}^{N} {w}_{i} \times {f}_{i} ( x )$$

In the formula above, N is the number of the identified plurality of sequence factors and N>1. fᵢ(x) denotes a penalty scoring function of the i-th sequence factor of the identified N sequence features; and wi denotes the relative weight given to fᵢ(x). Thus, the optimized gene should have low value of outlier index as far as possible.

In some embodiments, the plurality of sequence factors can be identified via one or more of steps 202, 204, and 208 shown in FIG. 2A. In some embodiments, the plurality of sequence factors contains, but not limited to, GC-content, CIS elements, repetitive elements, RNA splicing sites, ribosome binding sequences, minimal free energy of mRNA, described in detail below.

3(a). Minimal Free Energy (MFE) of mRNA

The potential strong stem-loop secondary structures of mRNA located in the downstream of the start codon may hinder the movement of the ribosome complex, and thus slow down the translation and reduce the translation efficiency. The steady secondary structures of mRNA can even cause the ribosome complex to fall off the mRNA and result in the premature termination of translation. There are several methods for free energy calculation and secondary structure prediction, including Mfold, RNAfold and RNAstructure. According to embodiments of the present invention, the local secondary structures of mRNA with a low free energy (ΔG<−18 Kcal/mol) or a long complementary stem (>10 bp) are defined as too stable for efficient translation. The gene sequences are preferably optimized to make the local structure not so stable. Both of the 5′-UTR and 3′-UTR of mRNA are preferably taken into consideration for mRNA structure free energy calculation and secondary structure prediction.

In some embodiments, the secondary structures that are considered too stable are associated with higher penalties. The weight used to give higher penalty score is flexible.

3(b). GC-Content

GC-content of mRNA is also preferably taken into account. An ideal range for GC % is approximately 30-70%. High GC-content will make mRNAs to form strong stem-loop secondary structures. It will also cause problems for PCR amplification and gene cloning. The high GC-content of the target sequence is preferably mutated (e.g., during the operation of the NSGA-III algorithm, including crossover and mutation of binary string) using codon degeneracy to be around 50-60%.

There are two different measurements for GC %. One is the global GC % which is averaged along the whole sequence; the other is more useful, which is the local GC % calculated within a shifted “window” of fixed size (e.g., 60 bp). According to embodiments of the present invention, the local GC % is optimized to around 35-65%.

3(c). Unstable Factors (e.g., Cis-acting mRNA Destabilizing Motifs, RNase Splicing Sites and Repetitive Element, etc.)

To reduce or minimize the mRNA degradation or increase the stability of mRNA thus to reduce the turnover time of mRNA, cis-acting mRNA destabilizing motifs including, but not limited to, AU-rich elements (AREs) and RNase recognition and cleavage sites is preferably mutated or deleted from the gene sequences. AU-rich elements (AREs) with the core motif of AUUUA (SEQ ID NO:1) are usually found in the 3′ untranslated regions of mRNA. Another example of the mRNA cis-element consists of sequence motif TGYYGATGYYYYY (SEQ ID NO:2), where Y stands for either T or C. RNase recognition sequences include, but are not limited to, RNase E recognition sequence. A host strain with deficient RNases can also be used for protein expression.

RNase splicing sites can cause RNA splicing to produce a different mRNA and therefore reduce the original mRNA level. RNase splicing sites are also preferably mutated to non-functional to maintain the mRNA level.

To produce high level of mRNA, the optimal transcription promoter sequence is preferably used in the gene sequences. For prokaryotic host such as E. coli, one of the strong promoters is T7 Promoter for T7 RNA Polymerase (T7 RNAP). Some bases of long or short tandem simple sequence repeat (SSR) are preferably mutated using codon degeneracy to break the repeats to reduce polymerase slippage, to thus reduce premature protein or protein mutations.

There are additional factors and parameters that affect mRNA translation and the resulting protein expression level. These factors affect translation from translation initiation through translation termination. Ribosomes bind mRNA at the ribosome binding site (RBS) to initiate translation. Because ribosomes do not bind to double-stranded RNA, the local mRNA structure around this region is preferably single Stranded and not form any stable secondary structure. The consensus RBS sequence, AGGAGG (SEQ ID NO:3), for prokaryotic cells such as E. coli, also called Shine-Dalgarnon sequence, is preferably placed a few bases just before the translation start site in the genes to be expressed. However, internal ribosome entry site (IRES) is preferably mutated to prevent ribosomes binding to avoid non-specific translation initiation.

Descriptions of the above-mentioned factors can be found in, for example, a publication titled “CIS/TRANSGENE OPTIMIZATION: SYSTEMATIC DISCOVERY OF NOVEL GENE EXPRESSION USING BIOINFORMATICS AND COMPUTATIONAL BIOLOGY APPROACHES” by Saeid Kadkhodaei et al., published in May 2018, a publication titled “AU-RICH ELEMENTS AND THE CONTROL OF GENE EXPRESSION THROUGH REGULATED MRNA STABILITY” by Timothy J Gingerich et al., published in July 2014, a publication titled “ARED-PLUS: AN UPDATED AND EXPANDED DATABASE OF AU-RICH ELEMENT-CONTAINING MRNAS AND PRE-MRNAS” by Tala Bakheet, published in October 2017, a publication titled “IDENTIFICATION AND CHARACTERIZATION OF A SEQUENCE MOTIF INVOLVED IN NONSENSE-MEDIATED MRNA DECAY” by Shuang Zhang et al., published in 1995, a publication titled “CORRELATIONS BETWEEN SHINE-DALGARNO SEQUENCES AND GENE FEATURES SUCH AS PREDICTED EXPRESSION LEVELS AND OPERON STRUCTURES” by Jiong Ma et al., published in 2002, a publication titled “AN INTERNAL RIBOSOME ENTRY SITE (IRES) MUTANT LIBRARY FOR TUNING EXPRESSION LEVEL OF MULTIPLE GENES IN MAMMALIAN CELLS” by Esther Y. C. Koh et al., published in December 2013, which are incorporated herein by reference in their entireties.

For various expression systems, the catalogues of adverse factors may change, of which the impacts or weights are also unequal. Thus the fᵢ(x) and its weight could be dynamically modified for various expression systems. For instance, after the setting of a permitted scope of GC-content and MFE, the extent of ‘out of range’ will cause penalty at the ratio. Likewise, the occurrence number of unstable factors may be directly recorded as the penalty scores.

It should be recognized that, even if the outlier index for a candidate nucleic acid sequence is high, the candidate sequence may still have some chance to survive the iteration so as to keep the diversity of whole population. In the other words, the adverse motifs/features filter through outlier index is not mandatory, because higher outlier index (i.e., penalty) can just result in a lower ratio of survival. In contrast, the removal of adverse motifs/features after the iterations of the NSGA-III algorithm are complete (i.e., in step 110 in FIG. 1 or step 214 in FIG. 2) is mandatory.

In conclusion, the invention not only attempts to promote positive effects by maximizing the values of harmony index and codon context index, but also tries its best to avoid adverse impact by minimizing the outlier index.

Multi-Objective (e.g., More Than 2 Objectives) Optimization Algorithm

As the present invention is an optimization task of three comprehensive objectives, a multi-objective genetic algorithm can be used. In some embodiments, the NSGA-III algorithm or its variants such as EliteNS GA-III (presented by K. Deb as well) can be used due to their advantages on solving many-objective optimization problem by maintaining the population diversity during the selection manipulation of classical framework of genetic algorithm.

NSGA-III was proposed by Kalyanmoy Deb and Himanshu Jain in 2014. It is a reference-point-based many-objective evolutionary algorithm following NSGA-II framework that emphasizes population members that are non-dominated, yet close to a set of supplied reference points. NSGA-III demonstrates its efficacy in solving three-objective to 15-objective optimization problems relative to other genetic algorithms, like NSGA-II. Unlike traditional genetic algorithm, the maintenance of diversity among population members in NSGA-III is aided by supplying and adaptively updating a number of well-spread predefined reference points, thus NSGA-III have significant changes in its selection operator.

The NSGA-III algorithm is described in a publication titled “An Evolutionary Many-Objective Optimization Algorithm Using Reference-Point-Based Nondominated Sorting Approach, Part I: Solving Problems With Box Constraints” by Kalyanmoy Deb et al., published in August 2014, which is incorporated herein by reference in its entirety. The related NSGA-II algorithm is described in a publication titled “A FAST AND ELITIST MULTIOBJECTIVE GENETIC ALGORITHM: NSGA-II” by Kalyanmoy Deb et al., published in August 2002, which is incorporated herein by reference in its entirety.

During the implementation of NSGA-III, binary string, but not codon list/array/vector, is selected as data structure to stand for nucleic acid sequences, and all general manipulation objects of general genetic algorithm including population initialization, crossover/recombination, mutation are binary strings, since binary string requires smaller computer memory and enables the faster manipulation speed relative to codon list/array/vector as data structure. In some embodiments, three continual bits are used to denote a codon at one position, since the number of all combination of three bits are enough to match all of the possible candidates of synonymous codons of certain amino acid. For instance, three bits have 8 kinds of combination, e.g., 000, 001, 010, 011, 100, 101, 110 and 111, of which the count is larger than the number of synonymous codons of any amino acid, even amino acid L, R and S which own 6 synonymous codons, respectively.

Thus, each one of 3 bit-strings stands for a synonymous codon of a given amino acid. During the fitness calculation (e.g., calculation of the harmony index, the codon context index, and the outlier index), a binary string standing for an individual candidate of the population is transformed back into the coding sequencing (i.e., DNA). On the other hand, as discussed above, the objects of operations (including crossover, mutation, selection) of genetic algorithm are all binary strings, thus the transformation is temporary. Thus, fitness calculations are based on sequences, while all of other operations are based on binary strings for efficiency and speed.

Before start of NSGA-III, a plurality of parameters are required to be set, including the size of population, the number of divisions, the distribution index for simulated binary crossover, the crossover rate for simulated binary crossover, the mutation rate for bit flip mutation, the distribution index for bit flip mutation. The authors of NSGA-III propose a two-layer approach for divisions for many-objective problems where an outer and inner division number is specified. To use the two-layer approach, we could replace the number of divisions with the number of outer divisions and the number of inner divisions. The initialization process of every individual is random, and crossover and mutation manipulation have no great difference with classical genetic algorithm shown in FIG. 2B.

FIG. 2B depicts an exemplary general workflow of genetic algorithm, including bio-inspired operators such as crossover, mutation and selection of population evolution. During the implementation of the present invention, binary string denotes a sequence therefore, the objects of all above operators are binary string.

When fitness functions (i.e., three index functions shown before) need to be evaluated for each individual of whole population before selection, the binary strings will be transferred back into codon strings temporally. After a number of evolution generations and the evolution termination, the finally generated codon strings will be concatenated and output as optimum genes used for recombinant expression.

In some embodiments, the terminating conditions include but are not limited to: fixed number of generations reached, best fitness reached a plateau and no better results produced, minimum criteria of near-optimal solution satisfied by some solutions.

According to the teachings of the NSGA-III algorithm, these optimum genes should be solutions located at pareto surface of three dimensional space and treated equally. For practical purposes, due to limited resource used for gene synthesis and expression test, we rank them by descending order of harmony index at first, then by descending order of codon context index and by ascending order of outlier index at last. The top 1 could be selected for synthesis and heterogenous expression given quota is only one sequence. Suppose there is no strict cost control, it is advised to test several of them which have enough interval at pareto surface, e.g., one candidate with highest harmony index, one candidate with highest codon context index and one candidate with lowest outlier index. In the present invention, the preliminary optimum genes have no stop codon, thus two continual stop codons could be appended at 3′ terminal of coding sequence.

Specific Subsequence Removal for Molecular Cloning

With reference to FIG. 2A, at block 214, the optimization procedure includes a step of motif avoidance and restriction site removal. With the aim to boost the convenience of molecular cloning, some adverse motifs and restriction site (e.g., those disliked by customers) are removed from one or more optimized sequences before gene synthesis and protein expression. The course contains:

Step 1: locating all subsequences which must be avoided.

Step 2: list all synonymous codons which could be used for substitution within a subsequence.

Step 3: the more frequently used synonymous codon within highly expressed genes have higher priority for selection on condition that we should keep no new subsequences emerge at the same time.

Step 4: iteratively deal with every found subsequence using step 2-3.

In some embodiments, as indicated in blocks 206 and 208, the adverse motifs and features are identified separately for various host by text mining and literature review.

Exemplary Realization

The exemplary realization described herein illustrates the efficiency of the present invention on codon optimization through the optimization and expression of two genes (JNK3A1 and GFP) at CHO 3E7 cell line, of which the basic information is summarized below. Since antibody of Flag tag was applied to perform western blot so as to evaluate the expression level, Flag tag was appended at C terminal of two proteins, meanwhile, beta-actin was used as the loading control. Each expression experiment was repeated twice.

| | GenBank | | | |
|---|---|---|---|---|
| | accession number | | Tag | |
| Protein | (Wild type) | Tag | location | Definition |
| JNK3A1 | U34820.1 | Flag | C- | Human JNK3 alpha1 |
| | | tag | terminal | protein kinase |
| GFP | L29345.1 | Flag | C- | Aequorea victoria green- |
| | | tag | terminal | fluorescent protein |

The mRNA-seq of CHO 3E7 cultured in several media including FreeStyle CHO Expression medium and CD CHO medium (Thermofish) were executed according to classical mRNA-seq proposal recommended by Illumina Integration with the partial orders successfully optimized of our company, totally 500 sequences were defined as highly expressed genes of CHO 3E7 cell line. After literature review, the following subsequences were grouped into adverse motifs, of which appearances resulted in penalty (i.e., increase of outlier index). The suitable local (60 bp sliding-window) and global GC-content are around 35-65%, and the acceptable minimum MFE ΔG of mRNA secondary structure is −18 Kcal/mol, outlier of these parameters caused the penalty.

1) Splice sites:
GGTAAG, GGTGAT
2) AT-rich elements:
ATTTTA, ATTTTTA, ATTTTTTA
3) Ribosome binding sites:
(SEQ ID NO: 4)
ACCACCATGG,
(SEQ ID NO: 5)
GCCACCATGG
4) Antiviral motifs:
TGTGT, AACGTT, CGTTCG, AGCGCT, GACGTC, GACGTT
5) CpG islands:
CGCGCGCG
6) Polymerase slippage site:
GGGGGG, CCCCCC
7) Amyloid precurser protein 3 prime stability
element:
(SEQ ID NO: 6)
TCTCTTTACATTTTGGTCTCTATACTACA
8) K-Box:
CTGTGATA
9) Brd-Box:
AGCTTTA

During codon optimization through NSGA-III, the population size was set to 100 and individual was binary encoded and randomly generated, of which the length equaled to the 3 folds of the number of amino acids of protein, the number of evolution generation equaled to 200,000, the number of divisions was dependent on the number of fitness functions, the distribution index for simulated binary crossover was 15.0, the single-point crossover rate for simulated binary crossover was 0.9, The mutation rate for bit flip mutation was 1.0/L, the distribution index for bit flip mutation was 20.0.

After maximizing the harmony index and codon context index alongside with minimizing the outlier index, each protein has several output optimum coding genes, of which only one gene had the maximum harmony index was selected for following expression test. Since EcoRI and HindIII enzyme were used for vector construction and cloning, GAATTC and AAGCTT were avoided by codon substitution.

The Sequence Listing submitted herein in the ASCII text file includes the optimized sequences of two proteins GFP_Flag (SEQ ID NO:7) and JNK3_Flag (SEQ ID NO:8).

Detailed steps of experiment used for evaluating the performance of optimized gene relative to wild type of the same gene is described below.

Step 1: Transient Transfection and Cell Culture

1. Synthesized gene was cloned into pTT5 vector using EcoRI and HindIII enzyme. CHO 3E7 cell was cultured in FreeStyle CHO Expression medium and transient transfection of vectors was done using standard molecular biology techniques with suitable cell-vector ratio (i.e., cell density 1-1.2×10⁶ per mL over vector concentration 1 ug/ml)

2. After transient transfection, CHO 3E7 cells required suspension culture in 37° C. with 5% CO₂, which lasted 48 hours.

Step 2: Cell Disruption

1. Get cultured cells from upstream, centrifuge(10,000×g) for 2 min at 4° C. Discard the supernatant.

2. Add 1 mL 1*PBS to resuspend cells at the bottom of the Eppendorf tube. Then centrifuge(10,000×g) for 2 min at 4° C. and discard the supernatant.

3. Add 200 μL Lysis Buffer (hypotonic buffer [10 mM Tris, 1.5 mM MgCl₂, 10 mM KCl, pH 7.9]+0.5% DDM, PMSF [final concentration 1 mM], nuclease, cocktail) into the Eppendorf tube per 1*10⁶ cells. Resuspend cells with pipette.

4. Place the cells in a cup-type ultrasonic cell disrupter for cell disruption (4° C., 3s ultrasound, 1s interval, 10 min totally).

5. After disruption, centrifuge(12,000×g) for 20 min at 4° C. Recover the supernatant.

Step 3: Sample Processing

1. Measure the concentration of supernatant using BCA method.

2. Part of supernatant was treated with loading buffer.

Step 4: Electrophoresis and Western Blot

1. Load the treated samples for SDS-PAGE according to SOP. (8 μg per sample)

2. After electrophoresis, Western Blot experiment was done according to SOP:

1) Transfer: Remove the gel after the SDS-PAGE, and transfer the protein from the gel to the PVDF membrane (transfer buffer: Add 200 mL 5× transfer solution to 150 mL of absolute ethanol and dilute to 1L, and transfer for 1 h).

2) Blocking: After the transfer, the PVDF was blocked with a fast blocking solution for 10 min.

3) Incubation: After blocking, incubate with 5% milk and corresponding labeled antibody for 45 min. (Flag tag: Mouse-anti-flag mAb GenScript, Cat.No.A00187 at a dilution of 1:5000, with addition of THETM beta Actin Antibody, mAb, Mouse GenScript, Cat.No.A00702 at a 1:1000 dilution for 1 h, then add a labeled secondary antibody Goat Anti-Mouse IgG-HRP GenScript, Cat.No.A00160 diluted 1:2500)

4) Exposure: Exposure imaging was performed using ChemiDoc™ Touch Imaging Systems after the antibody incubation, and the images are saved to a designated location for editing.

5) Image Lab was used for protein quantitative analysis.

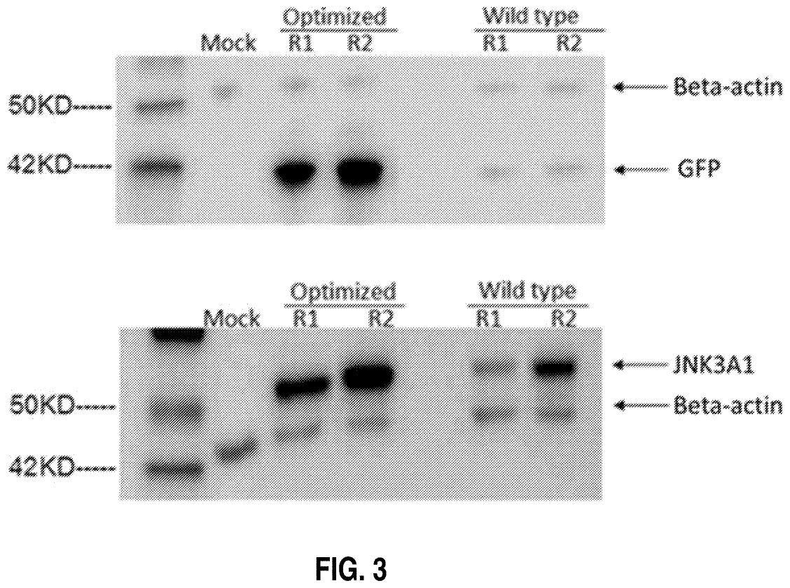

FIG. 3 is a western blot result, which illustrates a comparison of expressions between optimized sequence and wild type of two genes (i.e., GFP and JNK3A1) at CHO 3E7 cell line in accordance with an embodiment of the present disclosure, wherein only the optimized solution having highest harmony index of each gene was tested for expression comparison. It is obviously demonstrated that the invention is effective for codon optimization and boost the expression relative to almost unchanged internal control Beta-actin. The left lane was always ladder marker, and every expression of single plasmid was repeated twice. According to rough quantitative analysis, the expression of GFP was estimated to be improved approximately 6.2 fold, and the expression of JNK3 was promoted approximately 2.4 fold after codon optimization of this invention.

Exemplary Electronic Device

FIG. 4 illustrates an example of a computing device in accordance with one embodiment. Device 400 can be a host computer connected to a network. Device 400 can be a client computer or a server. As shown in FIG. 4, device 400 can be any suitable type of microprocessor-based device, such as a personal computer, workstation, server or handheld computing device (portable electronic device) such as a phone or tablet. The device can include, for example, one or more of processor 410, input device 420, output device 430, storage 440, and communication device 460. Input device 420 and output device 430 can generally correspond to those described above, and can either be connectable or integrated with the computer.

Input device 420 can be any suitable device that provides input, such as a touch screen, keyboard or keypad, mouse, or voice-recognition device. Output device 430 can be any suitable device that provides output, such as a touch screen, haptics device, or speaker.

Storage 440 can be any suitable device that provides storage, such as an electrical, magnetic or optical memory including a RAM, cache, hard drive, or removable storage disk. Communication device 460 can include any suitable device capable of transmitting and receiving signals over a network, such as a network interface chip or device. The components of the computer can be connected in any suitable manner, such as via a physical bus or wirelessly.

Software 450, which can be stored in storage 440 and executed by processor 410, can include, for example, the programming that embodies the functionality of the present disclosure (e.g., as embodied in the devices as described above).

Software 450 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 440, that can contain or store programming for use by or in connection with an instruction execution system, apparatus, or device.

Software 450 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 400 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 400 can implement any operating system suitable for operating on the network. Software 450 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

1. A computer-implemented method for optimizing a nucleic acid sequence for expression of a protein in a host, comprising:
a) receiving an initial population set, wherein the initial population set comprises a plurality of initial candidate nucleic acid sequences capable of expressing the protein; and
b) performing, based on the initial population set, optimization of a harmony index, a codon context index, and an outlier index using a computer-assisted NSGA-III algorithm or a variant thereof, thereby obtaining a plurality of optimized nucleic acid sequences capable of expressing the protein,
wherein the harmony index of a candidate nucleic acid sequence is indicative of consistency of usage frequency distribution of synonymous codons between a plurality of highly expressed genes and the candidate nucleic acid sequence,
wherein the codon context index of the candidate nucleic acid sequence is a measure for placing a synonymous codon into a suitable location, and
wherein the outlier index of the candidate nucleic acid sequence is a measure of negative effect of a plurality of predetermined sequence features on the candidate nucleic acid sequence.

2. The method according claim 1, further comprising providing an output indicative of at least one optimized nucleic acid sequence of the plurality of optimized nucleic acid sequences.

3. The method of claim 1, wherein receiving an initial population set comprises:
receiving a protein sequence;
generating the initial population set based on the received protein sequence.

4. The method of claim 1, wherein receiving an initial population set comprises:
receiving a nucleic acid sequence;
translating the received nucleic acid sequence into a protein sequence;
generating the initial population set based on the protein sequence.

5. (canceled)

6. (canceled)

7. The method of claim 1, wherein performing optimization of a harmony index, a codon context index, and an outlier index comprises:
maximizing the harmony index;
maximizing the codon context index; and
minimizing the outlier index.

8. (canceled)

9. (canceled)

10. (canceled)

11. (canceled)

12. (canceled)

13. (canceled)

14. (canceled)

15. (canceled)

16. The method according to claim 1, wherein the harmony index of a candidate nucleic acid sequence is calculated based on a formula: H=1−D(Fₕₛ,Fₜₛ),
wherein D( ) indicates a distance function;
wherein Fₕₛ includes a vector comprising frequencies of synonymous codons of a plurality of amino acids within a plurality of highly expressed genes; and
wherein Fₜₛ includes a vector comprising of frequencies of synonymous codons of the plurality of amino acids within a coding gene of the candidate nucleic acid sequence.

17. (canceled)

18. The method of claim 17, wherein D( ) is a distance function that includes, but is not limited to: Euclidean distance, a Cosine distance, a Manhattan distance, or a Minkowski distance of two vectors.

19. The method according to claim 18, wherein a frequency of a synonymous codon of the plurality of highly expressed genes or a candidate nucleic acid sequence is defined as:
$${F}_{{s}_{ij}} = \frac{total occurancy of synonymous codon j}{total occurancy of amino acid i} , ∀ i ∈ \{ A , C , D , E , F , G , H , I , K , L , N , P , Q , R , S , T , V , Y \} and ∃ j ∈ 59 synonymous codons .$$

20. The method according to claim 1, wherein the codon context index of a candidate nucleic acid sequence is calculated based on a formula: CC=1−D(F_{hcc},F_{tcc}),
wherein D( ) indicates a distance function;
wherein F_{hcc} comprises a vector comprising frequencies of synonymous codon pairs of two continual amino acids within a plurality of highly expressed genes; and
wherein F_{hcc} comprises a vector comprising frequencies of synonymous codon pairs of two continual amino acids within a coding gene of the candidate nucleic acid sequence.

21. (canceled)

22. The method of claim 21, wherein D( ) is a distance function that includes, but is not limited to: Euclidean distance, a Cosine distance, a Manhattan distance, or a Minkowski distance of two vectors.

23. The method according to claim 20, wherein a frequency of a synonymous codon pair of the plurality of highly expressed genes or a candidate nucleic acid sequence is defined as:
$${F}_{{cc}_{ij}} = \frac{total occurancy of synonymous codon pair j}{total occurancy of amino acid pair i} , ∀ i ∈ the permutation of two amino acids besides MM , MW , WW and WM \text{;} ∃ j ∈ 3717 codon pairs .$$

24. The method according to claim 1, wherein the outlier index is calculated based on a formula: O=Σᵢ₌₁^{N} wᵢ×fᵢ(x),
wherein N is the number of the plurality of predetermined sequence features;
wherein fi(x) denotes a penalty scoring function of the ith sequence feature of the plurality of predetermined sequence features; and
wherein wi denotes a relative weight associated with fi(x).

25. The method according to claim 24, wherein the plurality of predetermined features includes:
GC-content value,
CIS elements,
repetitive elements,
RNA splicing sites,
ribosome binding sequences,
minimal free energy of mRNA, or
any combination thereof.

26. (canceled)

27. The method according to claim 1, wherein a variant of the NSGA-III algorithm includes the EliteNS GA-III algorithm or a NSGA-II based immune algorithm.

28. The method according to claim 1, wherein performing optimization of a harmony index, a codon context index, and an outlier index comprises:
ranking the plurality of optimized nucleic acid sequences by descending order of harmony index, then by descending order of codon context index, and then by ascending order of outlier index;
selecting one or more top-ranked optimized nucleic acid sequences for synthesis.

29. The method according to claim 1, further comprising:
c) removing a predetermined adverse subsequence or motif from an optimized nucleic acid sequence of the plurality of optimized nucleic acid sequences.

30. (canceled)

31. The method according to claim 29, wherein removing the predetermined adverse subsequence or motif comprises:
identifying the predetermined adverse subsequence or motif in the optimized nucleic acid sequence;
identifying a plurality of synonymous codons based on identified predetermined adverse subsequence or motif;
selecting a synonymous codon from the plurality of synonymous codons for substitution with the identified predetermined adverse subsequence in the optimized nucleic acid sequence.

32. The method according to claim 1, wherein at least one of the harmony index, the codon context index, and the outlier index is calculated based on one or more characteristics of a plurality of highly-expressed genes from one or more databases.

33. The method according to claim 32, wherein the one or more characteristics include codon frequency, synonymous codon frequency, codon pair frequency, or a combination thereof.

34. (canceled)

35. (canceled)

36. A system for optimizing a nucleic acid sequence for expression of a protein in a host, the system comprising:
one or more processors;
a memory; and
one or more programs, wherein the one or more programs are stored in the memory and configured to be executed by the one or more processors, the one or more programs including instructions for carrying out the method of claim 1.

37.-42. (canceled)
